(19) United States
(12) Patent Application Publication
(10) Pub. No.: US 2015/0320650 A1
(43) Pub. Date: Nov. 12, 2015

(54) ANHYDROUS COSMETIC COMPOSITION

(71) Applicant: L'OREAL, Paris (FR)

(72) Inventors: Arno WAHLER, Vincennes (FR); Maxime DE BONI, Tokyo (JP); Anne-Laure BERNARD, New York, NY (US); Jinglan LI, Tokyo (JP)

(21) Appl. No.: 14/653,315

(22) PCT Filed: Dec. 18, 2013
(86) PCT No.: PCT/JP2013/084770

§ 371 Date: Jun. 18, 2015

(30) Foreign Application Priority Data
Dec. 19, 2012 (JP) 2012-276748

(51) Int. Cl. A61K 8/03; A61K 8/34; A61Q 5/12; A61K 8/86; A61Q 1/14; A61K 8/31; A61K 8/39

(52) U.S. Cl. CPC A61K 8/03; A61K 8/31; A61K 8/345; A61K 8/39; A61K 8/86; A61Q 1/14; A61Q 5/12; A61K 2800/31; A61K 2800/805

(57) ABSTRACT

The present invention relates to an anhydrous cosmetic composition, comprising: (a) at least one oil phase including at least one oil; (b) at least one alcohol phase including at least one alcohol; and (c) at least one nonionic surfactant phase including at least one nonionic surfactant, wherein the amount of the (c) nonionic surfactant phase is less than 10% by weight relative to the total weight of the composition. The anhydrous cosmetic composition according to the present invention can provide multi-phase appearance, and can visually stimulate users of the cosmetic composition while maintaining good cosmetic effects.

### TECHNICAL FIELD

The present invention relates to an anhydrous cosmetic composition which can be separated into a plurality of liquid phases which are visually distinct

### BACKGROUND ART

Oils are commonly used in cosmetics in order to, for example, provide conditioning effects for skin or hair, or remove a make-up.

For hair, oils are well known to provide softness and brightness to the hair. However, the feeling to touch provided by the application of oils onto hair may not be preferable. Thus, hair cosmetics for conditioning the hair are often based on emulsions which include water and some conditioning agents such as oils, as well as, typically, cationic agents. Due to the anionic nature of the hair, some cationic agents are electrochemically adsorbed on the hair to provide softness to the hair. Also, hair cosmetic for deterging the hair includes emulsions which include water and some surfactants which can solublize sebum on the hair by incorporating it into micelles.

For skin, rinse-off skin care oils are often used for body to provide moisture to the skin. However, they are often difficult to use because they are too fluidable. In addition, the rinsibility of the rinse-off skin care oils is not always sufficient. Furthermore, they are expensive. Also, cleansing oils are used in particular in Asian countries to remove eye and face make-up. However, they have the same problems, because they are too fluidable, they may be difficult to be rinsed off in some cases, and they are relatively expensive.

### DISCLOSURE OF INVENTION

In typical cosmetics for hair and skin, at least one oil as a conditioner is combined with at least one surfactant as an emulsifier or detergent, and with a polar phase such as water. This combination often results in an emulsion which has a classical aspect and texture such as white and creamy.

On the other hand, there is a need to stimulate potential consumers visually.

An objective of the present invention is to provide a cosmetic composition which can stimulate visually potential consumers while it can provide good cosmetic effects such as conditioning and cleansing effects, and can be easily rinsed off. The cosmetic composition according to the present invention can give non-dry final feeling after the application thereof, while giving moisturized feeling.

The above objective can be achieved by an anhydrous cosmetic composition comprising:
(a) at least one oil phase including at least one oil;
(b) at least one alcohol phase including at least one alcohol; and
(c) at least one nonionic surfactant phase including at least one nonionic surfactant, wherein
   the amount of the (c) nonionic surfactant phase is less than 10% by weight relative to the total weight of the composition.

It is preferable that at least two of the three phases, and most preferably the three phases, i.e., the (a) oil phase, the (b) alcohol phase, and the (c) nonionic surfactant phase in the anhydrous cosmetic composition according to the present invention be visually distinct after coming in a state of rest.

It is more preferable that the (c) nonionic surfactant phase in the anhydrous cosmetic composition according to the present invention be present between the (a) oil phase and the (b) alcohol phase.

The oil may be selected from the group consisting of aliphatic hydrocarbons, preferably mineral oils.

The amount of the (a) oil phase may range from 10 to 80% by weight, preferably from 20 to 70% by weight, and more preferably from 30 to 60% by weight, relative to the total weight of the composition.

The alcohol may be selected from polyols, preferably glycerins and derivatives thereof, and glycols and derivatives thereof. It is more preferable that the polyol be selected from the group consisting of glycerin, diglycerin, polyglycerin, ethyleneglycol, diethyleneglycol, propyleneglycol, dipropyleneglycol, butyleneglycol, pentyleneglycol, hexyleneglycol, 1,3-propanediol, and 1,5-pentanediol.

The amount of the (b) alcohol phase may range from 5 to 80% by weight, preferably from 8 to 70% by weight, and more preferably from 10 to 60% by weight, relative to the total weight of the composition.

The nonionic surfactant may have an HLB value of 18.0 or less, preferably from 4.0 to 18.0, more preferably from 6.0 to 15.0, and even more preferably from 9.0 to 13.0.

The nonionic surfactant may be selected from esters of polyols with fatty acids with a saturated or unsaturated chain containing for example from 8 to 24 carbon atoms, preferably 12 to 22 carbon atoms, and alkoxylated derivatives thereof, such as glyceryl esters of a C₈-C₂₄ fatty acid or acids and alkoxylated derivatives thereof, polyethylene glycol esters of a C₈-C₂₄ fatty acid or acids and alkoxylated derivatives thereof, sorbitol esters of a C₈-C₂₄ fatty acid or acids and alkoxylated derivatives thereof, sugar (sucrose, glucose, alkylglycose) esters of a C₈-C₂₄ fatty acid or acids and alkoxylated derivatives thereof, ethers of fatty alcohols, ethers of sugar and a C₈-C₂₄ fatty alcohol or alcohols, and mixtures thereof.

It is preferable that the nonionic surfactant be selected from the group consisting of PEG-7 glyceryl cocoate, PEG-20 methylglucoside sesquistearate, PEG-20 glyceryl tri-isostearate, PG-5 dioleate, PG-4 diisostearate, PG-10 isostearate, PEG-8 isostearate, and PEG-60 hydrogenated castor oil.

The amount of the (c) nonionic surfactant phase may range from 0.1 to 9% by weight, preferably from 1 to 8.5% by weight, and more preferably from 5 to 8% by weight, relative to the total weight of the composition.

The anhydrous cosmetic composition according to the present invention may further comprise water in an amount of 1% by weight or less relative to the total weight of the composition.

The present invention also relates to a cosmetic process for keratin substance comprising the step of applying the anhydrous cosmetic composition according to the present invention to dry or wet keratin substance such as hair and skin.

It is preferable that the cosmetic process for keratin substance according to the present invention comprise the step of mixing the (a) oil phase, the (b) alcohol phase and the (c) nonionic surfactant phase in the anhydrous cosmetic composition before the step of applying the anhydrous cosmetic composition to the keratin substance.

### BEST MODE FOR CARRYING OUT THE INVENTION

After diligent research, the inventors have discovered that it is possible to provide a cosmetic composition which can stimulate visually potential consumers while it can provide good cosmetic effects such as conditioning and cleansing effects, and can be easily rinsed off.

Thus, the cosmetic composition according to the present invention is anhydrous and comprises:
(a) at least one oil phase including at least one oil;
(b) at least one alcohol phase including at least one alcohol; and
(c) at least one nonionic surfactant phase including at least one nonionic surfactant, wherein
   the amount of the (c) nonionic surfactant phase is less than 10% by weight relative to the total weight of the composition.

The anhydrous cosmetic composition according to the present invention can have multiple liquid phases. Each liquid phase can provide any independent visual effect. Thus, the multi liquid phases can provide unique appearances to the anhydrous cosmetic composition according to the present invention.

On the other hand, the multiple liquid phases can disappear when mixing them by, for example, shaking the anhydrous cosmetic composition according to the present invention to form a uniform phase. Thus, for example, if the refractive indexes of the multiple liquid phases are similar to each other, it is possible for the uniform phase to be transparent. The uniform phase can separate into the multiple liquid phases again by leaving it, without any shear force, in a certain period of time such as from few minutes to several hours.

Hereafter, each of the phases constituting the anhydrous cosmetic composition according to the present invention will be described in a detailed manner.

### [Oil Phase]

The cosmetic composition according to the present invention includes at least one oil phase including at least one oil. Two or more oils may be used in combination. Thus, a single type of oil or a combination of different type of oils may be used. The oil phase is in the form of a liquid at ambient temperature such as 25° C. under atmospheric pressure (760 mmHg or 10⁵ Pa).

Preferably, the oil used in the present invention does not contain a polyalkylenated or polygycerolated group or a salified carboxylic group.

The oil may be selected from the group consisting of oils of animal or plant origin, mineral oils, synthetic glycerides, esters of fatty alcohols and/or fatty acids other than animal or plant oils and synthetic glycerides, fatty alcohols, non salified fatty acids, silicone oils and aliphatic hydrocarbons. These fatty materials may be volatile or non-volatile. It is preferable that the oil be selected from aliphatic hydrocarbons, plant oils, fatty alcohols, esters of fatty alcohols and/or fatty acids other than animal or plant oils and synthetic glycerides, or mixtures thereof. It is more preferable that the oil be selected from the group consisting of aliphatic hydrocarbons, in particular mineral oils.

As examples of aliphatic hydrocarbons, mention may be made of, for example, linear or branched hydrocarbons such as mineral oils (e.g., liquid paraffin), paraffin, vaseline or petrolatum, naphthalenes, and the like; hydrogenated polyisobutene, isoeicosan, polydecenes, hydrogenated polyisobutenes such as Parleam, and decene/butene copolymer; and mixtures thereof.

As examples of other aliphatic hydrocarbons, mention may also be made of linear or branched, or possibly cyclic C₆-C₁₆ lower alkanes. Examples that may be mentioned include hexane, undecane, dodecane, tridecane and isoparaffins such as isohexadecane and isodecane.

As example of synthetic glycerides, mention may be made of, for instance, caprylic/capric acid triglycerides, for instance those sold by the company Stéarineries Dubois or those sold under the names Miglyol® 810, 812 and 818 by the company Dynamit Nobel.

As examples of silicone oils, mention may be made of, for example, linear organopolysiloxanes such as dimethylpolysiloxane, methylphenylpolysiloxane, methylhydrogenpolysiloxane, and the like; cyclic organopolysiloxanes such as octamethylcyclotetrasiloxane, decamethylcyclopentasiloxane, dodecamethylcyclohexasiloxane, and the like; and mixtures thereof.

As examples of plant oils, mention may be made of, for example, linseed oil, camellia oil, macadamia nut oil, sunflower oil, apricot oil, soybean oil, arara oil, hazelnut oil, corn oil, mink oil, olive oil, avocado oil, sasanqua oil, castor oil, safflower oil, jojoba oil, sunflower oil, almond oil, grapeseed oil, sesame oil, soybean oil, peanut oil, and mixtures thereof.

As examples of animal oils, mention may be made of, for example, squalene, perhydrosqualene and squalane.

As examples of the esters of fatty alcohols and/or of fatty acids, which are advantageously different from the animal or plant oils as well as the synthetic glycerides mentioned above, mention may be made especially of esters of saturated or unsaturated, linear or branched C₁-C₂₆ aliphatic mono- or polyacids and of saturated or unsaturated, linear or branched C₁-C₂₆ aliphatic mono- or polyalcohols, the total carbon number of the esters being greater than or equal to 10.

Among the monoesters, mention may be made of dihydroabietyl behenate; octyldodecyl behenate; isocetyl behenate; cetyl lactate; C₁₂-C₁₅ alkyl lactate; isostearyl lactate; lauryl lactate; linoleyl lactate; oleyl lactate; (iso)stearyl octanoate; isocetyl octanoate; octyl octanoate; cetyl octanoate; decyl oleate; isocetyl isostearate; isocetyl laurate; isocetyl stearate; isodecyl octanoate; isodecyl oleate; isononyl isononanoate; isostearyl palmitate; methylacetyl ricinoleate; myristyl stearate; octyl isononanoate; 2-ethylhexyl isononate; octyl palmitate; octyl pelargonate; octyl stearate; octyldodecyl erucate; oleyl erucate; ethyl and isopropyl palmitates, 2-ethylhexyl palmitate, 2-octyldecyl palmitate, alkyl myristates such as isopropyl, butyl, cetyl, 2-octyldodecyl, myristyl or stearyl myristate, hexyl stearate, butyl stearate, isobutyl stearate; dioctyl malate, hexyl laurate, 2-hexyldecyl laurate.

Still within the context of this variant, esters of C₄-C₂₂ dicarboxylic or tricarboxylic acids and of C₁-C₂₂ alcohols and esters of mono-, di- or tricarboxylic acids and of C₂-C₂₆ di-, tri-, tetra- or pentahydroxy alcohols may also be used.

The following may especially be mentioned: diethyl sebacate; diisopropyl sebacate; diisopropyl adipate; di-n-propyl adipate; dioctyl adipate; diisostearyl adipate; dioctyl maleate; glyceryl undecylenate; octyldodecyl stearoyl stearate; pentaerythrityl monoricinoleate; pentaerythrityl tetraisononanoate; pentaerythrityl tetrapelargonate; pentaerythrityl tetraisostearate; pentaerythrityl tetraoctanoate; propylene glycol dicaprylate; propylene glycol dicaprate; tridecyl erucate; triisopropyl citrate; triisostearyl citrate; glyceryl trilactate; glyceryl trioctanoate; trioctyldodecyl citrate; trioleyl citrate; propylene glycol dioctanoate; neopentyl glycol diheptanoate; diethylene glycol diisononanoate; and polyethylene glycol distearates.

Among the esters mentioned above, it is preferred to use ethyl, isopropyl, myristyl, cetyl or stearyl palmitate, 2-ethylhexyl palmitate, 2-octyldecyl palmitate, alkyl myristates such as isopropyl, butyl, cetyl or 2-octyldodecyl myristate, hexyl stearate, butyl stearate, isobutyl stearate; dioctyl malate, hexyl laurate, 2-hexyldecyl laurate, isononyl isononanoate or cetyl octanoate.

The oil phase may also comprise, as fatty ester, sugar esters and diesters of C₆-C₃₀ and preferably C₁₂-C₂₂ fatty acids. It is recalled that the term “sugar” means oxygen-bearing hydrocarbon-based compounds containing several alcohol functions, with or without aldehyde or ketone functions, and which contain at least 4 carbon atoms. These sugars may be monosaccharides, oligosaccharides or polysaccharides.

Examples of suitable sugars that may be mentioned include sucrose (or saccharose), glucose, galactose, ribose, fructose, maltose, mannose, arabinose, xylose and lactose, and derivatives thereof, especially alkyl derivatives, such as methyl derivatives, for instance methylglucose.

The sugar esters of fatty acids may be chosen especially from the group comprising the esters or mixtures of esters of sugars described previously and of linear or branched, saturated or unsaturated C₆-C₃₀ and preferably C₁₂-C₂₂ fatty acids. If they are unsaturated, these compounds may comprise one to three conjugated or non-conjugated carbon-carbon double bonds.

The esters according to this variant may also be chosen from mono-, di-, tri-, tetraesters and polyesters, and mixtures thereof.

These esters may be chosen, for example, from oleates, laurates, palmitates, myristates, behenates, cocoates, stearates, linoleates, linolenates, caprates and arachidonates, or mixtures thereof such as, especially, oleo-palmitate, oleo-stearate and palmito-stearate mixed esters.

It is more particularly preferred to use monoesters and diesters and especially sucrose, glucose or methylglucose mono- or dioleates, stearates, behenates, oleopalmitates, linoleates, linolenates and oleostearates.

An example that may be mentioned is the product sold under the name Glucate® DO by the company Amerchol, which is a methylglucose dioleate.

Examples of esters or mixtures of esters of sugar and of fatty acid that may also be mentioned include:
the products sold under the names F160, F140, F110, F90, F70 and SL40 by the company Crodesta, respectively denoting sucrose palmitostearates formed from 73% monoester and 27% diester and triester, from 61% monoester and 39% diester, triester and tetraester, from 52% monoester and 48% diester, triester and tetraester, from 45% monoester and 55% diester, triester and tetraester, from 39% monoester and 61% diester, triester and tetraester, and sucrose monolaurate;
the products sold under the name Ryoto Sugar Esters, for example referenced B370 and corresponding to sucrose behenate formed from 20% monoester and 80% di-triester-polyester;
the sucrose mono-dipalmito-stearate sold by the company Goldschmidt under the name Tegosoft® PSE.

The oil phase may include be at least one fatty acid. Two or more fatty acids may be used. The fatty acids should be in acidic form (i.e., unsalified, to avoid soaps) and may be saturated or unsaturated and contain from 6 to 30 carbon atoms and in particular from 9 to 30 carbon atoms, which is optionally substituted, in particular with one or more hydroxyl groups (in particular 1 to 4). If they are unsaturated, these compounds may comprise one to three conjugated or non-conjugated carbon-carbon double bonds. They are more particularly chosen from myristic acid, palmitic acid, stearic acid, behenic acid, oleic acid, linoleic acid, linolenic acid and isostearic acid. Preferably the fatty material is not a fatty acid.

The oil phase may include at least one fatty alcohol, and two or more fatty alcohols may be used.

The term “fatty alcohol” here means any saturated or unsaturated, linear or branched C₈-C₃₀ fatty alcohol, which is optionally substituted, in particular with one or more hydroxyl groups (in particular 1 to 4). If they are unsaturated, these compounds may comprise one to three conjugated or non-conjugated carbon-carbon double bonds. Preferably fatty alcohols are unsaturated and/or branched.

Among the C₈-C₃₀ fatty alcohols, C₁₂-C₂₂ fatty alcohols, for example, are used. Mention may be made among these of, isostearyl alcohol, oleyl alcohol, linoleyl alcohol, undecylenyl alcohol, palmitoleyl alcohol, linolenyl alcohol, erucyl alcohol, and mixtures thereof. In one embodiment, cetyl alcohol, stearyl alcohol or a mixture thereof (e.g., cetearyl alcohol), as well as myristyl alcohol, can be used as a solid fatty material. In one embodiment, isostearyl alcohol can be used as a liquid fatty material.

The oil phase may include at least one wax. Here, “wax” means a fatty compound substantially in the form of a solid at room temperature (25° C.) under atmospheric pressure (760 mmHg), and has a melting point generally of 35° C. or more. As the waxy fatty material, waxes generally used in cosmetics can be used alone or in combination thereof.

The wax may be a fatty alcohol.

In one embodiment, cetyl alcohol, stearyl alcohol or a mixture thereof (e.g., cetearyl alcohol), as well as myristyl alcohol, can be used as the solid fatty material.

The wax may be also chosen from carnauba wax, microcrystalline waxes, ozokerites, hydrogenated jojoba oil, polyethylene waxes such as the wax sold under the name “Performalene 400 Polyethylene” by the company New Phase Technologies, silicone waxes, for instance poly(C₂₄-C₂₈)alkylmethyldimethylsiloxane, such as the product sold under the name “Abil Wax 9810” by the company Goldschmidt, palm butter, the C₂₀-C₄₀ alkyl stearate sold under the name “Kester Wax K82H” by the company Kester Keunen, stearyl benzoate, shellac wax, and mixtures thereof. For example, a wax chosen from carnauba wax, candelilla wax, ozokerites, hydrogenated jojoba oil and polyethylene waxes is used. In at least one embodiment, the wax is preferably chosen from candelilla wax and ozokerite, and mixtures thereof.

The oil phase may include at least one colorant, but it is preferable that the oil phase be transparent or translucent.

It is preferable that the oil phase is free from silicone(s). The term “free from” here means that the oil phase may contain only a small amount of silicone(s), preferably no silicone(s). Thus, the amount of silicone(s) may be 5% by weight or less, preferably 3% by weight or less, more preferably 1% by weight or less of silicone(s). It is particularly preferable that the oil phase contains no silicone(s).

The oil phase may include at least one lipophilic compound such as oil-soluble organic or inorganic compounds (e.g., some types of amino acids). The amount of the lipophilic compound is 50% by weight or less relative to the total weight of the oil phase. Thus, the amount of oil(s) in the oil phase may range from 50 to 100% by weight, preferably 60 to 100% by weight, and more preferably from 70 to 100% by weight, relative to the total weight of the oil phase.

The amount of the (a) oil phase may range from 10 to 80% by weight, preferably from 20 to 70% by weight, and more preferably from 30 to 60% by weight, relative to the total weight of the composition.

### [Alcohol Phase]

The cosmetic composition according to the present invention includes at least one alcohol phase including at least one liquid alcohol. Two or more alcohols may be used in combination. Thus, a single type of alcohol or a combination of different type of alcohols may be used. The alcohol phase is in the form of a liquid at ambient temperature such as 25° C. under atmospheric pressure (760 mmHg or 10⁵ Pa).

The term “alcohol” here means an alcohol having one, two or more hydroxy groups, and does not encompass a saccharide or a derivative thereof. The derivative of a saccharide includes a sugar alcohol which is obtained by reducing one or more carbonyl groups of a saccharide, as well as a saccharide or a sugar alcohol in which the hydrogen atom or atoms in one or more hydroxy groups thereof has or have been replaced with at least one substituent such as an alkyl group, a hydroxyalkyl group, an alkoxy group, an acyl group or a carbonyl group.

Alcohols used in the present invention are liquid at ambient temperature such as 25° C. under atmospheric pressure (760 mmHg or 10⁵ Pa).

When the alcohol has one hydroxy group, it is a monoalcohol.

When the alcohol has two or more hydroxy groups, it is a polyol.

The monoalcohol is preferably a C₂-C₂₄ monoalcohol, more preferably a C₂-C₁₂ linear or branched monoalcohol, most preferably a C₂-C₄ linear or branched monoalcohol. Preferred monoalcohols are ethanol and isopropanol.

The polyol may be a C₂₋₂₄ polyol, preferably a C₂₋₉ polyol, comprising at least 2 hydroxy groups, and preferably 2 to 5 hydroxy groups.

The polyol may be a natural or synthetic polyol. The polyol may have a linear, branched or cyclic molecular structure.

The polyol may be selected from glycerins and derivatives thereof, and glycols and derivatives thereof. The polyol may be selected from the group consisting of glycerin, diglycerin, polyglycerin, ethyleneglycol, diethyleneglycol, propyleneglycol, dipropyleneglycol, butyleneglycol, pentyleneglycol, hexyleneglycol, C₆-C₂₄ polyethyleneglycol; 1,3-propanediol, 1,4-butanediol, and 1,5-pentanediol.

The alcohol phase may optionally include an alkylene oxide derivative represented by the following formula (I):

Z-{O(AO)ₗ(EO)ₘ—(BO)ₙH}ₐ (I)

wherein
Z denotes a residue obtained by removing fully (or totally) hydroxyl group(s) from a compound having 3 to 9 hydroxyl groups;
AO denotes an oxyalkylene group having 3 to 4 carbon atoms;
EO denotes an oxyethylene group;
BO denotes an oxyalkylene group having 4 carbon atoms;
a denotes 3 to 9;
l, m, and n denote the average addition mole numbers of AO, EO and BO, respectively, and 1≦l≦50, 1≦m≦50 and 0.5≦n≦5;
a weight ratio of AO to EO (AO/EO) ranges from 1/5 to 5/1; and
AO and EO may have been added randomly or in the form of blocks.

The aforementioned alkylene oxide derivatives may be a single type thereof or a mixture of plural types thereof.

In the alkylene oxide derivative represented by formula (I), Z denotes a residue obtained by fully removing hydroxyl groups from a compound having 3 to 9 hydroxyl groups, and a denotes the number of hydroxyl groups of the compound and is 3 to 9. As examples of compounds having 3 to 9 hydroxyl groups, mention may be made of, for example, in the case of a=3, glycerin, and trimethylolpropane; in the case of a=4, erythritol, pentaerythritol, sorbitol, alkylglycosides, and diglycerin; in the case of a=5, xylitol; in the case of a=6, dipentaerythritol, sorbitol, and inositol; in the case of a=8, sucrose, and trehalose; in the case of a=9, maltitol; mixtures thereof; and the like. Preferably, Z denotes a residue obtained by removing hydroxyl group(s) from a compound having 3 to 6 hydroxyl groups, and a satisfies 3≦a≦6. As the compound having 3 to 9 hydroxyl groups, glycerin or trimethylolpropane is preferable, and in particular, glycerin is preferable. In the case of a≦2, poor compatibility with oil components such as fats and oils is exhibited, and blending stability in an oil-based formulation tends to be impaired. In the case of 10≦a, stickiness occurs.

AO denotes an oxyalkylene group having 3 to 4 carbon atoms. As examples thereof, mention may be made of, for example, an oxypropylene group, an oxybutylene group (an oxy-n-butylene group, an oxyisobutylene group, or an oxy-t-butylene group), an oxytrimethylene group, an oxytetramethylene group, and the like. The oxypropylene group and oxybutylene group are preferable, and the oxypropylene group is more preferable.

l denotes the average addition mole number of AO, and satisfies 1≦l≦50, and preferably 2≦l≦20. m denotes the average addition mole number of EO, and satisfies 1≦m≦50, and preferably 2≦m≦20. If l is 0, stickiness occurs. On the other hand, if l exceeds 50, moisturizing effects are decreased. In addition, if m is 0, moisturizing effects are decreased. On the other hand, if m exceeds 50, stickiness occurs.

The weight ratio of AO to EO (AO/EO) ranges from 1/5 to 5/1, and preferably ranges from 1/4 to 4/1. If AO/EO is below 1/5, stickiness occurs. On the other hand, if AO/EO exceeds 5/1, the moisturizing sensation is decreased. The order of adding AO and EO is not particularly specified. AO and EO can be added randomly or in the form of blocks. In order to obtain superior effects of preventing skin roughness, AO and EO are preferably added randomly.

BO denotes an oxyalkylene group having 4 carbon atoms. As examples thereof, mention may be made of, for example, an oxybutylene group (an oxy-n-butylene group, an oxy-isobutylene group, or an oxy-t-butylene group), an oxytetramethylene group, and the like. The oxybutylene group is preferable.

n denotes the average addition mole number of BO, and satisfies 0.5<n≦5, preferably 0.8≦n≦3, and more preferably 1≦n≦3. If n is below 0.5, stickiness occurs. On the other hand, if n exceeds 5, moisturizing effects are decreased. In formula (I), it is necessary that (BO)ₙ bonds to the terminal hydrogen atom.

The alkylene oxide derivatives represented by formula (I) can be produced by means of known methods. For example, the alkylene oxide derivatives represented by formula (I) can be obtained by additive-polymerizing ethylene oxide and an alkylene oxide having 3 to 4 carbon atoms to a compound having 3 to 9 hydroxyl groups, and subsequently reacting with an alkylene oxide having 4 carbon atoms. When additive-polymerizing ethylene oxide and an alkylene oxide having 3 to 4 carbon atoms to a compound having 3 to 9 hydroxyl groups, the ethylene oxide and alkylene oxide may be polymerized randomly or in the form of blocks.

Among the alkylene oxide derivatives represented by formula (I), preferable examples of the aforementioned alkylene oxide derivatives include, for example, an alkylene oxide derivative (polyoxybutylene polyoxyethylene polyoxypropylene glycerol) represented by formula (II) shown below:

Gly-[O(PO)ₛ(EO)ₜ—(BO)ᵤH]₃ (II)

wherein
Gly denotes a residue obtained by removing hydroxyl groups from glycerin;
PO denotes an oxypropylene group;
EO denotes an oxyethylene group;
s and t denote the average addition mole numbers of PO and EO, respectively, and have a value ranging from 1 to 50;
the weight ratio of PO to EO (PO/EO) ranges from 1/5 to 5/1;
BO denotes an oxyalkylene group having 4 carbon atoms; and
u denotes the average addition mole number of BO, and ranges from 0.5 to 5.

The aforementioned alkylene oxide derivative represented by formula (II) can be obtained by adding propylene oxide and ethylene oxide to glycerin, in the ratio of 3 to 150 mole equivalents of each of propylene oxide and ethylene oxide with respect to glycerin, and subsequently, adding the alkylene oxide having 4 carbon atoms in the ratio of 1.5 to 15 mole equivalents thereof with respect to glycerin.

In the case of adding the aforementioned alkylene oxides to glycerin, the addition reactions are carried out with an alkali catalyst, a phase transfer catalyst, a Lewis acid catalyst, or the like. In general, an alkali catalyst such as potassium hydroxide is preferably employed.

Among the alkylene oxide derivatives represented by formula (I), more preferable derivatives are obtained by adding 6 to 10 mol of ethylene oxide and 3 to 7 mol of propylene oxide to glycerin, and subsequently, adding 2 to 4 mol of butylene oxide.

Among the alkylene oxide derivatives represented by formula (I), a further more preferable derivative is polyoxybutylene polyoxyethylene polyoxypropylene glycerol, which is obtained by adding 8 mol of ethylene oxide and 5 mol of propylene oxide to glycerin, and subsequently, adding 3 mol of butylene oxide, and which has an INCI name of PEG/PPG/polybutylene glycol-8/5/3 glycerin. PEG/PPG/polybutylene glycol-8/5/3 glycerin is commercially available under the trade name of WILBRIDE S-753 from NOF Corporation.

The alcohol phase may include at least one colorant, but it is preferable that the alcohol phase be transparent or translucent.

The alcohol phase may include at least one compound which is soluble in the alcohol phase such as some types of amino acids, nonionic active ingredients such as Vitamin C, and nonionic surfactant with an HLB value of more than 13.0. The amount of the alcohol-soluble compound is 50% by weight or less relative to the total weight of the alcohol phase. In particular, the amount of the nonionic surfactant with an HLB value of more than 13.0 is preferably 3% by weight or less, and more preferably 2% by weigh or less, and furthermore preferably 1% by weight or less, relative to the total weight of the alcohol phase. Thus, the amount of alcohol(s) in the alcohol phase may range from 50 to 100% by weight, preferably 60 to 100% by weight, and more preferably from 70 to 100% by weight, relative to the total weight of the alcohol phase.

The amount of the (b) alcohol phase may range from 5 to 80% by weight, preferably from 8 to 70% by weight, and more preferably from 10 to 60% by weight, relative to the total weight of the composition.

### [Nonionic Surfactant Phase]

The cosmetic composition according to the present invention includes at least one nonionic surfactant phase including at least one nonionic surfactant. Two or more nonionic surfactants may be used in combination. Thus, a single type of nonionic surfactant or a combination of different type of nonionic surfactants may be used. The nonionic surfactant phase is in the form of a liquid at ambient temperature such as 25° C. under atmospheric pressure (760 mmHg or 10⁵ Pa).

The nonionic surfactants are compounds well known in themselves (see, e.g., in this regard, “Handbook of Surfactants” by M. R. Porter, Blackie & Son publishers (Glasgow and London), 1991, pp. 116-178). Thus, they can, for example, be chosen from alcohols, alpha-diols, alkylphenols and esters of fatty acids, these compounds being ethoxylated, propoxylated or glycerolated and having at least one fatty chain comprising, for example, from 8 to 18 carbon atoms, it being possible for the number of ethylene oxide or propylene oxide groups to range from 2 to 50, and for the number of glycerol groups to range from 1 to 30. Maltose derivatives may also be mentioned. Non-limiting mention may also be made of copolymers of ethylene oxide and/or of propylene oxide; condensates of ethylene oxide and/or of propylene oxide with fatty alcohols; polyethoxylated fatty amides comprising, for example, from 2 to 30 mol of ethylene oxide; polyglycerolated fatty amides comprising, for example, from 1.5 to 5 glycerol groups, such as from 1.5 to 4; ethoxylated fatty acid esters of sorbitan comprising from 2 to 30 mol of ethylene oxide; ethoxylated oils from plant origin; fatty acid esters of sucrose; fatty acid esters of polyethylene glycol; polyethoxylated fatty acid mono or diesters of glycerol (C₆-C₂₄)alkylpolyglycosides; N—(C₆-C₂₄)alkylglucamine derivatives, amine oxides such as (C₁₀-C₁₄)alkylamine oxides or N—(C₁₀-C₁₄)acylaminopropylmorpholine oxides; and mixtures thereof.

The nonionic surfactants may preferably be chosen from polyoxyalkylenated or polyglycerolated nonionic surfactants. The oxyalkylene units are more particularly oxyethylene or oxypropylene units, or a combination thereof, and are preferably oxyethylene units.

Examples of oxyalkylenated nonionic surfactants that may be mentioned include:
oxyalkylenated (C₈-C₂₄)alkylphenols,
saturated or unsaturated, linear or branched, oxyalkylenated C₈-C₃₀ alcohols,
saturated or unsaturated, linear or branched, oxyalkylenated C₈-C₃₀ amides,
esters of saturated or unsaturated, linear or branched, C₈-C₃₀ acids and of polyethylene glycols, polyoxyalkylenated esters of saturated or unsaturated, linear or branched, C₈-C₃₀ acids and of sorbitol,
saturated or unsaturated, oxyalkylenated plant oils,
condensates of ethylene oxide and/or of propylene oxide, inter alia, alone or as mixtures.

The surfactants preferably contain a number of moles of ethylene oxide and/or of propylene oxide of between 2 and 100 and most preferably between 2 and 50. Advantageously, the nonionic surfactants do not comprise any oxypropylene units.

In accordance with one preferred embodiment of the invention, the oxyalkylenated nonionic surfactants are chosen from oxyethylenated C₈-C₃₀ alcohols.

Examples of ethoxylated fatty alcohols (or C₈-C₃₀ alcohols) that may be mentioned include the adducts of ethylene oxide with lauryl alcohol, especially those containing from 9 to 50 oxyethylene groups and more particularly those containing from 10 to 12 oxyethylene groups (Laureth-10 to Laureth-12, as the CTFA names); the adducts of ethylene oxide with behenyl alcohol, especially those containing from 9 to 50 oxyethylene groups (Beheneth-9 to Beheneth-50, as the CTFA names); the adducts of ethylene oxide with cetearyl alcohol (mixture of cetyl alcohol and stearyl alcohol), especially those containing from 10 to 30 oxyethylene groups (Ceteareth-10 to Ceteareth-30, as the CTFA names); the adducts of ethylene oxide with cetyl alcohol, especially those containing from 10 to 30 oxyethylene groups (Ceteth-10 to Ceteth-30, as the CTFA names); the adducts of ethylene oxide with stearyl alcohol, especially those containing from 10 to 30 oxyethylene groups (Steareth-10 to Steareth-30, as the CTFA names); the adducts of ethylene oxide with isostearyl alcohol, especially those containing from 10 to 50 oxyethylene groups (Isosteareth-10 to Isosteareth-50, as the CTFA names); and mixtures thereof.

As examples of polyglycerolated nonionic surfactants, polyglycerolated C₈-C₄₀ alcohols are preferably used.

In particular, the polyglycerolated C₈-C₄₀ alcohols correspond to the following formula:

RO—[CH₂—CH(CH₂OH)—O]ₘ—H or RO—[CH(CH₂OH)—CH₂O]ₘ—H

in which R represents a linear or branched C₈-C₄₀ and preferably C₈-C₃₀ alkyl or alkenyl radical, and m represents a number ranging from 1 to 30 and preferably from 1.5 to 10.

As examples of compounds that are suitable in the context of the invention, mention may be made of lauryl alcohol containing 4 mol of glycerol (INCI name: Polyglyceryl-4 Lauryl Ether), lauryl alcohol containing 1.5 mol of glycerol, oleyl alcohol containing 4 mol of glycerol (INCI name: Polyglyceryl-4 Oleyl Ether), oleyl alcohol containing 2 mol of glycerol (INCI name: Polyglyceryl-2 Oleyl Ether), cetearyl alcohol containing 2 mol of glycerol, cetearyl alcohol containing 6 mol of glycerol, oleocetyl alcohol containing 6 mol of glycerol, and octadecanol containing 6 mol of glycerol.

The alcohol may represent a mixture of alcohols in the same way that the value of m represents a statistical value, which means that, in a commercial product, several species of polyglycerolated fatty alcohol may coexist in the form of a mixture.

According to one of the embodiments according to the present invention, the nonionic surfactant may be selected from esters of polyols with fatty acids with a saturated or unsaturated chain containing for example from 8 to 24 carbon atoms, preferably 12 to 22 carbon atoms, and alkoxylated derivatives thereof, preferably with a number of alkyleneoxide of from 10 to 200, and more preferably from 10 to 100, such as glyceryl esters of a C₈-C₂₄, preferably C₁₂-C₂₂, fatty acid or acids and alkoxylated derivatives thereof, preferably with a number of alkyleneoxide of from 10 to 200, and more preferably from 10 to 100; polyethylene glycol esters of a C₈-C₂₄, preferably C₁₂-C₂₂, fatty acid or acids and alkoxylated derivatives thereof, preferably with a number of alkyleneoxide of from 10 to 200, and more preferably from 10 to 100; sorbitol esters of a C₈-C₂₄, preferably C₁₂-C₂₂, fatty acid or acids and alkoxylated derivatives thereof, preferably with a number of alkyleneoxide of from 10 to 200, and more preferably from 10 to 100; sugar (sucrose, glucose, alkylglycose) esters of a C₈-C₂₄, preferably C₁₂-C₂₂, fatty acid or acids and alkoxylated derivatives thereof, preferably with a number of alkyleneoxide of from 10 to 200, and more preferably from 10 to 100; ethers of fatty alcohols; ethers of sugar and a C₈-C₂₄, preferably C₁₂-C₂₂, fatty alcohol or alcohols; and mixtures thereof.

Examples of ethoxylated fatty esters that may be mentioned include the adducts of ethylene oxide with esters of lauric acid, palmitic acid, stearic acid or behenic acid, and mixtures thereof, especially those containing from 9 to 100 oxyethylene groups, such as PEG-9 to PEG-50 laurate (as the CTFA names: PEG-9 laurate to PEG-50 laurate); PEG-9 to PEG-50 palmitate (as the CTFA names: PEG-9 palmitate to PEG-50 palmitate); PEG-9 to PEG-50 stearate (as the CTFA names: PEG-9 stearate to PEG-50 stearate); PEG-9 to PEG-50 palmitostearate; PEG-9 to PEG-50 behenate (as the CTFA names: PEG-9 behenate to PEG-50 behenate); polyethylene glycol 100 EO monostearate (CTFA name: PEG-100 stearate); and mixtures thereof.

As glyceryl esters of fatty acids, glyceryl stearate (glyceryl mono-, di- and/or tristearate) (CTFA name: glyceryl stearate) or glyceryl ricinoleate and mixtures thereof can in particular be cited.

As glyceryl esters of C₈-C₂₄ alkoxylated fatty acids, polyethoxylated glyceryl stearate (glyceryl mono-, di- and/or tristearate) such as PEG-20 glyceryl stearate can for example be cited.

Mixtures of these surfactants, such as for example the product containing glyceryl stearate and PEG-100 stearate, marketed under the name ARLACEL 165 by Uniqema, and the product containing glyceryl stearate (glyceryl mono- and distearate) and potassium stearate marketed under the name TEG1N by Goldschmidt (CTFA name: glyceryl stearate SE), can also be used.

The sorbitol esters of C₈-C₂₄ fatty acids and alkoxylated derivatives thereof can be selected from sorbitan palmitate, sorbitan trioleate and esters of fatty acids and alkoxylated sorbitan containing for example from 20 to 100 EO, such as for example polyethylene sorbitan trioleate (polysorbate 85) or the compounds marketed under the trade names Tween 20 or Tween 60 by Ubiqema.

As esters of fatty acids and glucose or alkylglucose, in particular glucose palmitate, alkylglucose sesquistearates such as methylglucose sesquistearate, alkylglucose palmitates such as methylglucose or ethylglucose palmitate, methylglucoside fatty esters and more specifically the diester of methylglucoside and oleic acid (CTFA name: Methyl glucose dioleate), the mixed ester of methylglucoside and the mixture oleic acid/hydroxystearic acid (CTFA name: Methyl glucose dioleate/hydroxystearate), the ester of methylglucoside and isostearic acid (CTFA name: Methyl glucose isostearate), the ester of methylglucoside and lauric acid (CTFA name: Methyl glucose laurate), the mixture of monoester and diester of methylglucoside and isostearic acid (CTFA name: Methyl glucose sesqui-isostearate), the mixture of monoester and diester of methylglucoside and stearic acid (CTFA name: Methyl glucose sesquistearate) and in particular the product marketed under the name Glucate SS by AMERCHOL, and mixtures thereof can be cited.

As ethoxylated ethers of fatty acids and glucose or alkylglucose, ethoxylated ethers of fatty acids and methylglucose, and in particular the polyethylene glycol ether of the diester of methylglucose and stearic acid with about 20 moles of ethylene oxide (CTFA name: PEG-20 methyl glucose distearate) such as the product marketed under the name Glucam E-20 distearate by AMERCHOL, the polyethylene glycol ether of the mixture of monoester and diester of methyl-glucose and stearic acid with about 20 moles of ethylene oxide (CTFA name: PEG-20 methyl glucose sesquistearate) and in particular the product marketed under the name Glucamate SSE-20 by AMERCHOL and that marketed under the name Grillocose PSE-20 by GOLDSCHMIDT, and mixtures thereof, can for example be cited.

As sucrose esters, saccharose palmito-stearate, saccharose stearate and saccharose monolaurate can for example be cited.

As sugar ethers, alkylpolyglucosides can be used, and for example decylglucoside such as the product marketed under the name MYDOL 10 by Kao Chemicals, the product marketed under the name PLANTAREN 2000 by Henkel, and the product marketed under the name ORAMIX NS 10 by Seppic, caprylyl/capryl glucoside such as the product marketed under the name ORAMIX CG 110 by Seppic or under the name LUTENSOL GD 70 by BASF, laurylglucoside such as the products marketed under the names PLANTAREN 1200 N and PLANTACARE 1200 by Henkel, coco-glucoside such as the product marketed under the name PLANTACARE 818/UP by Henkel, cetostearyl glucoside possibly mixed with cetostearyl alcohol, marketed for example under the name MONTANOV 68 by Seppic, under the name TEGO-CARE CG90 by Goldschmidt and under the name EMULGADE KE3302 by Henkel, arachidyl glucoside, for example in the form of the mixture of arachidyl and behenyl alcohols and arachidyl glucoside marketed under the name MONTANOV 202 by Seppic, cocoylethylglucoside, for example in the form of the mixture (35/65) with cetyl and stearyl alcohols, marketed under the name MONTANOV 82 by Seppic, and mixtures thereof can in particular be cited.

Mixtures of glycerides of alkoxylated plant oils such as mixtures of ethoxylated (200 EO) palm and copra (7 EO) glycerides can also be cited.

It is preferable that the nonionic surfactant be selected from the group consisting of PEG-7 glyceryl cocoate, PEG-20 methylglucoside sesquistearate, PEG-20 glyceryl tri-isostearate, PG-5 dioleate, PG-4 diisostearate, PG-10 isostearate, PEG-8 isostearate, and PEG-60 hydrogenated castor oil.

Mixtures of these oxyethylenated derivatives of fatty alcohols and of fatty esters may also be used.

Preferably, the nonionic surfactant may be a nonionic surfactant with an HLB of 18.0 or less, such as from 4.0 to 18.0, more preferably from 6.0 to 15.0 and furthermore preferably from 9.0 to 13.0. The HLB is the ratio between the hydrophilic part and the lipophilic part in the molecule. This term HLB is well known to those skilled in the art and is described in “The HLB system. A time-saving guide to emulsifier selection” (published by ICI Americas Inc., 1984).

It is preferable that the nonionic surfactant is soluble in the oil phase and/or the alcohol phase.

The amount of the (c) nonionic surfactant phase is less than 10% by weight relative to the total weight of the composition, and may range from 0.1 to 9% by weight, preferably from 1 to 8.5% by weight, and more preferably from 5 to 8% by weight, relative to the total weight of the composition. If the amount of the (c) nonionic surfactant is 10% by weigh or more relative to the total weight of the composition, the cosmetic composition according to the present invention may give inferior cosmetic effects in terms of moisturizing keratin substance such as skin and hair.

### [Cosmetic Composition]

The cosmetic composition according to the present invention is anhydrous. The term “anhydrous” here means that the cosmetic composition according to the present invention may contain only a small amount of water, preferably no water. Thus, the amount of water may be 2% by weight or less, preferably 1.5% by weight or less, more preferably 1% by weight or less relative to the total weight of the composition. It is particularly preferable that the cosmetic composition according to the present invention contains no water.

According to the present invention, the (a) oil phase, the (b) alcohol phase, and the (c) nonionic surfactant phase in the anhydrous cosmetic composition can directly contact to each other. Typically, the above three phases may be packaged in a single container.

It is preferable that the (a) oil phase, the (b) alcohol phase, and the (c) nonionic surfactant phase in the anhydrous cosmetic composition according to the present invention can be spontaneously separated from each other. Preferably, at least two of the three phases are visually distinct after coming in a state of rest. More preferably, the three phases can be separated. This phase separation can visually stimulate the users of the anhydrous cosmetic composition according to the present invention. It is more preferable that the (c) nonionic surfactant phase in the anhydrous cosmetic composition according to the present invention can be present between the (a) oil phase and the (b) alcohol phase. The phase separation of the anhydrous cosmetic composition according to the present invention can be caused by leaving it without any shear force for a certain period of time such as 10 minutes to 24 hours.

It is preferable that the anhydrous cosmetic composition according to the present invention can form a homogeneous phase when being mixed by, for example, shaking the composition by hand. It is preferable that the homogeneous phase be in the lamellar form, and viscous such that it does not drip off.

The viscosity of the anhydrous cosmetic composition according to the present invention is not particularly limited. The viscosity can be measured at 25° C. with viscosimeters or rheometers preferably with cone-plane geometry. Preferably, the viscosity of the anhydrous cosmetic composition according to the present invention can range, for example, from 1 to 2000 Pa·s, and preferably from 1 to 1000 Pa·s at 25° C. and 1 s⁻¹.

The anhydrous cosmetic composition according to the present invention may also comprise an effective amount of cosmetic additives such as anionic, cationic or amphoteric surfactants, thickeners, sequestering agents, UV screening agents, preserving agents, vitamins or provitamins, opacifiers, fragrances, plant extracts, humectants, waxes, fillers, colouring materials, antioxydants, proteins, and so on.

If any of the (a) oil phase, the (b) alcohol phase, and the (c) nonionic surfactant phase is colored, the anhydrous cosmetic composition according to the present invention may be more visually attractive for users.

The anhydrous cosmetic composition according to the present invention can be prepared by mixing the above essential or optional components by using a conventional mixing means such as a mixer and a homogenizer.

The anhydrous cosmetic composition according to the present invention can be used as cosmetics for skin or hair, such as hair rinse-off or leave on products (e.g., shampoos and conditioners), make-up removers (e.g., cleansing products), make-up products (e.g., foundations), body wash products, and skin moisturizing products.

Since the anhydrous cosmetic composition according to the present invention is constituted by not only oil(s) but also alcohol(s), and nonionic surfactant(s), it can be prepared by using a less amount of oils as compared to conventional oil-based cosmetic products, and therefore, this can reduce the cost to prepare the anhydrous cosmetic composition according to the present invention.

### [Cosmetic Process]

The anhydrous cosmetic composition according to the present invention can be used in a cosmetic process for keratin substance comprising the step of applying the anhydrous cosmetic composition according to the present invention to keratin substance. The keratin substance here means a material containing keratin as a main constituent element, and examples thereof include skin, nails, lips, hair and the like.

The keratin substance can be in a dry state or in a wet state before application of the cosmetic composition according to the present invention. The application of the cosmetic composition according to the invention to the keratin substance may or may not be followed by rinsing the keratin substance. Before rinsing, the cosmetic composition according to the present invention can be left in contact with the keratin substance, for example, from 30 seconds to 30 minutes.

It is preferable that the cosmetic process for keratin substance according to the present invention comprise the step of mixing the (a) oil phase, the (b) alcohol phase and the (c) nonionic surfactant phase in the anhydrous cosmetic composition, for example by hand, before the step of applying the anhydrous cosmetic composition to the keratin substance.

By mixing the anhydrous cosmetic composition according to the present invention, a homogeneous phase is formed. Since the homogeneous phase includes oil(s), alcohol(s) and nonionic surfactant(s) equally, the cosmetic effects provided by these ingredients can be provided uniformly to the keratin substance, which will result in good balance of cosmetic effects provided by each of the phases (a) to (c).

Since the homogeneous phase is not in the form of an O/W emulsion, oil and/or alcohol (they are capable of forming a layer on keratin substance which can inhibit the evaporation of water from the keratin substance, while they can solubilize hydrophobic or hydrophilic substances used in, for example, make-up cosmetics) can directly contact the keratin substance, and therefore, the anhydrous cosmetic composition according to the present invention can provide superior moisturizing and/or cleansing effects.

Furthermore, due to the presence of the nonionic surfactant(s), the anhydrous cosmetic composition according to the present invention can be easily rinsed off. In particular, when the anhydrous cosmetic composition according to the present invention is used with water, it can easily form an emulsion, and can be smoothly removed from the keratin substance. The alcohol can also be removed easily, when the anhydrous cosmetic composition according to the present invention is used with water, because of its hydrophilic nature.

In addition, as compared to anionic, cationic or amphoteric surfactants, nonionic surfactants are less irritative, and therefore, the anhydrous cosmetic composition according to the present invention can provide good feeling to use.

### EXAMPLES

The present invention will be described in more detail by way of examples, which however should not be construed as limiting the scope of the present invention.

### Example 1

An anhydrous cosmetic composition was prepared by mixing the ingredients shown in Table 1. The mixture was filled into a transparent glass tube container. After vigorous shaking by hand, the anhydrous cosmetic composition according to Example 1 was in the form of a homogeneous phase. About 10 minutes after the shaking, it was observed that the anhydrous cosmetic composition separated into 3 phases.

### Comparative Example 1

An anhydrous cosmetic composition was prepared by mixing the ingredients shown in Table 1. The mixture was filled into a transparent glass tube container. After vigorous shaking by hand, the anhydrous cosmetic composition according to Comparative Example 1 was in the form of a homogeneous phase. About 10 minutes after the shaking, it was observed that the anhydrous cosmetic composition was separated into 3 phases.

### [Evaluation of Cosmetic Effects]

The compositions according to Example 1 and Comparative Example 1 were evaluated as follows: They were applied on the make-up on the face, at room temperature (25° C.); then, they were rinsed with water stream.

The specific evaluation protocol is as follows:
The quantity of the make-up (liquid foundation) applied was 0.01 g/cm².
The cosmetic composition was applied with finger on the area of 2×2 cm².
The quantity of the cosmetic composition was 0.2 g.
Number of circles rubbing was 30.
The rinsing was performed under tap water.
Excess water was removed by slight padding with a paper sheet and let dry.

The results of the evaluation results are shown in Table 1.

TABLE 1

| | Example 1 | Comparative Example 1 |
|---|---|---|
| Mineral Oil | 31 wt % | 15 wt % |
| PEG-7 Glyceryl Cocoate | 8 wt % | 24 wt % |
| Glycerin | 60 wt % | 60 wt % |
| Ethylhexyl methoxycinnamate | 0.3 wt % | 0.3 wt % |
| Fragrance | 0.7 wt % | 0.7 wt % |
| Make-up Removal | Very Good | Very Good |
| Dripping Down | Same | Same |
| Rinsing | Very Good | Very Good |
| Finishing | Not Dry | Dry |

As a make-up remover, the composition according to Example 1 showed very good removability of the make-up on the skin, and very good rinsability. Furthermore, there was no dripping down when applying the composition according to Example 1 on the face, while the composition according to Comparative Example 1 showed dripping down.

Advantageously, the composition according to Example 1 provided non-dry final sensation (final feeling), while providing sensation of moisturized skin; that is not the case with the composition according to Comparative Example 1.

### Examples 2 to 8

The following haircare compositions were prepared by mixing the ingredients shown in Table 2 (% by weight).

TABLE 2

| | Ex. | Ex. | Ex. | Ex. | Ex. | Ex. | Ex. |
|---|---|---|---|---|---|---|---|
| | 2 | 3 | 4 | 5 | 6 | 7 | 8 |
| Mineral Oil | 49 | 46 | 45 | 40.8 | 40.8 | 40.8 | 40.8 |
| (liquid paraffin) | | | | | | | |
| Polyquaternium 22 | — | — | 1 | — | — | — | — |
| Polyethyleneglycol 8OE | — | — | — | 34.8 | 34.8 | 34.8 | 34.8 |
| Ethanol | 10 | 5 | 5 | — | — | — | — |
| Glycerol | 40 | 45 | 44 | 15 | 15.33 | 14 | 14.33 |
| Cetrimonium chloride | — | — | — | — | — | 1 | 1 |
| PEG-20 METHYL | 1 | 4 | — | — | — | — | — |
| GLUCOSE | | | | | | | |
| SESQUISTEARATE | | | | | | | |
| PEG-20 GLYCERYL | — | — | 5 | 8.9 | 8.9 | 8.9 | 8.9 |
| TRIISOSTEARATE | | | | | | | |

### [Evaluation of Cosmetic Effects]

The compositions according to Examples 2 to 8 were evaluated as follows: They were applied on the tip and middle of wet hair just after being subjected to shampooing, as a rinse-off conditioner, at room temperature (25° C.), followed by soft massaging the hair; then, they were rinsed with water stream. After the rinse step, the hair was dried with a drier or with a towel in the atmosphere at ambient temperature. The feeling to touch the hair was evaluated. During the rinse step, the compositions according to Examples 4, 5 and 6 was in the appearance of milk. In comparison with the use of pure oil on the same conditions, the feeling to touch of the hair treated with the compositions according to Examples 2 to 8 was not oily but moisture feel.

### Examples 9 to 11

The following skincare compositions were prepared by mixing the ingredients shown in Table 3 (% by weight).

TABLE 3

| | Ex. 9 | Ex. 10 | Ex. 11 |
|---|---|---|---|
| Vaselin Oil | — | — | 5 |
| Mineral Oil | 40.8 | 40.8 | 35 |
| (Liquid Paraffin) | | | |
| Polyethylene glycol 8 OE | 34.8 | 34.8 | 35 |
| Glycerol | 15 | 15.33 | 20 |
| PEG-20 GLYCERYL | 8.9 | 8.9 | 5 |
| TRIISOSTEARATE | | | |

### [Evaluation of Cosmetic Effects]

The compositions according to Examples 9 to 11 were evaluated as follows: They were applied on the make-up on the face, at room temperature (25° C.); then, they were rinsed with water stream.

The specific evaluation protocol is as follows:
The quantity of the make-up (liquid foundation) applied was 0.01 g/cm².
The cosmetic composition was applied with finger on the area of 2×2 cm².
The quantity of the cosmetic composition was 0.2 g.
Number of circles rubbing was 30.
The rinsing was performed under tap water.
Excess water was removed by slight padding with a paper sheet and let dry.

As a make-up remover, the compositions according to Examples 9 to 11 showed good removability of the make-up on the skin, and good rinsability. Furthermore, there was no dripping down when applying the composition according to Examples 9 to 11 on the face.

The above evaluations were performed on 12 women.

The compositions according to Examples 9 to 11 were easy to homogenize and the resulting texture was gelified and translucent which makes the application of the compositions to the skin easy (no dripping). Further, waterproof mascara and foundation were easily removed, and rinsing was good. Furthermore, there was no eye discomfort.

1.-15. (canceled)

16. An anhydrous cosmetic composition, comprising:
(a) at least one oil phase including at least one oil;
(b) at least one alcohol phase including at least one alcohol; and
(c) at least one nonionic surfactant phase including at least one nonionic surfactant,
wherein the total amount of the nonionic surfactant phase is less than about 10% by weight, relative to the total weight of the composition.

17. The anhydrous cosmetic composition according to claim 16, wherein the at least one oil phase, the at least one alcohol phase, and the at least one nonionic surfactant phase are chosen to be capable of being separated from each other.

18. The anhydrous cosmetic composition according to claim 17, wherein the at least one nonionic surfactant phase is present between the at least one oil phase and the at least one alcohol phase when the at least one oil phase, the at least one alcohol phase, and the at least one nonionic surfactant phase are separated from each other.

19. The anhydrous cosmetic composition according to claim 16, wherein the at least one oil is chosen from aliphatic hydrocarbons.

20. The anhydrous cosmetic composition according to claim 16, wherein the at least one oil is chosen from mineral oils.

21. The anhydrous cosmetic composition according to claim 16, wherein the total amount of the oil phase ranges from about 10% to about 80% by weight, relative to the total weight of the composition.

22. The anhydrous cosmetic composition according to claim 16, wherein the total amount of the oil phase ranges from about 30% to about 60% by weight, relative to the total weight of the composition.

23. The anhydrous cosmetic composition according to claim 16, wherein the at least one alcohol is chosen from polyols, glycerins or derivatives thereof, glycols or derivatives thereof, or mixtures thereof.

24. The anhydrous cosmetic composition according to claim 23, wherein the at least one polyol is chosen from glycerin, diglycerin, polyglycerin, ethyleneglycol, diethyleneglycol, propyleneglycol, dipropyleneglycol, butyleneglycol, pentyleneglycol, hexyleneglycol, 1,3-propanediol, 1,5-pentanediol, or mixtures thereof.

25. The anhydrous cosmetic composition according to claim 16, wherein the total amount of the alcohol phase ranges from about 5% to about 80% by weight, relative to the total weight of the composition.

26. The anhydrous cosmetic composition according to claim 16, wherein the total amount of the alcohol phase ranges from about 10% to about 60% by weight, relative to the total weight of the composition.

27. The anhydrous cosmetic composition according to claim 16, wherein the at least one nonionic surfactant has an HLB value of about 18.0 or less.

28. The anhydrous cosmetic composition according to claim 16, wherein the at least one nonionic surfactant has an HLB value ranging from about 9.0 to about 13.0.

29. The anhydrous cosmetic composition according to claim 16, wherein the at least one nonionic surfactant is chosen from esters of polyols with fatty acids with a saturated or unsaturated chain, or alkoxylated derivatives thereof; esters of polyols with fatty acids with a saturated or unsaturated chain containing from 8 to 24 carbon atoms, or alkoxylated derivatives thereof; glyceryl esters of a C₈-C₂₄ fatty acid or acids, or alkoxylated derivatives thereof; polyethylene glycol esters of a C₈-C₂₄ fatty acid or acids, or alkoxylated derivatives thereof; sorbitol esters of a C₈-C₂₄ fatty acid or acids, or alkoxylated derivatives thereof; sugar esters of a C₈-C₂₄ fatty acid or acids, or alkoxylated derivatives thereof; sucrose esters of a C₈-C₂₄ fatty acid or acids, or alkoxylated derivatives thereof; glucose esters of a C₈-C₂₄ fatty acid or acids, or alkoxylated derivatives thereof; alkylglycose esters of a C₈-C₂₄ fatty acid or acids, or alkoxylated derivatives thereof; ethers of fatty alcohols; ethers of sugar and a C₈-C₂₄ fatty alcohol or alcohols; or mixtures thereof.

30. The anhydrous cosmetic composition according to claim 16, wherein the at least one nonionic surfactant is chosen from PEG-7 glyceryl cocoate, PEG-20 methylglucoside sesquistearate, PEG-20 glyceryl tri-isostearate, PG-5 dioleate, PG-4 diisostearate, PG-10 isostearate, PEG-8 isostearate, PEG-60 hydrogenated castor oil, or mixtures thereof.

31. The anhydrous cosmetic composition according to claim 16, wherein the total amount of the nonionic surfactant phase ranges from about 0.1% to about 9% by weight, relative to the total weight of the composition.

32. The anhydrous cosmetic composition according to claim 16, wherein the total amount of the nonionic surfactant phase ranges from about 5% to about 8% by weight, relative to the total weight of the composition.

33. The anhydrous cosmetic composition according to claim 16, further comprising water in an amount of about 1% by weight or less, relative to the total weight of the composition.

34. A cosmetic process for treating dry or wet keratin substance, the process comprising:
applying to the keratin substance an anhydrous cosmetic composition comprising:
(a) at least one oil phase including at least one oil;
(b) at least one alcohol phase including at least one alcohol; and
(c) at least one nonionic surfactant phase including at least one nonionic surfactant,
wherein the total amount of the nonionic surfactant phase is less than about 10% by weight, relative to the total weight of the composition.

35. The cosmetic process according to claim 34, further comprising:
mixing the at least one oil phase, the at least one alcohol phase, and the at least one nonionic surfactant phase in the anhydrous cosmetic composition prior to applying the anhydrous cosmetic composition to the keratin substance.
